(19) United States
(12) Patent Application Publication
(10) Pub. No.: US 2021/0372947 A1
(43) Pub. Date: Dec. 2, 2021

(54) METHODS AND MEANS FOR IDENTIFYING FLUID TYPE INSIDE A CONDUIT

(71) Applicant: Visuray Intech Ltd (BVI), Road Town (VG)

(72) Inventors: Kambiz Safinya, Houston, TX (US); Melissa Spannuth, Houston, TX (US); Spencer Gunn, London (GB)

(73) Assignee: Visuray Intech Ltd (BVI), Road Town (VG)

(21) Appl. No.: 17/398,727

(22) Filed: Aug. 10, 2021

Related U.S. Application Data
(63) Continuation of application No. 16/185,720, filed on Nov. 9, 2018.
Application No. 16/185,720 is a division of application No. 15/144,047, filed on May 2, 2016, now abandoned.
(60) Provisional application No. 62/154,955, filed on Apr. 30, 2015.

(51) Int. Cl. G01N 23/00; G01N 33/28; G01V 5/12; E21B 47/113; E21B 49/08; G01N 33/24

(52) U.S. Cl. CPC G01N 23/00; G01N 33/2823; G01V 5/125; G01N 23/203; E21B 49/08; G01N 33/241; E21B 47/113

(57) ABSTRACT

An x-ray-based borehole fluid evaluation tool for evaluating the characteristics of a fluid located external to said tool in a borehole using x-ray backscatter imaging is disclosed, the tool including at least an x-ray source; a radiation shield to define the output faun of the produced x-rays into the borehole fluid outside of the tool housing; at least one collimated imaging detector to record x-ray backscatter images; sonde-dependent electronics; and a plurality of tool logic electronics and power supply units. A method of using an x-ray-based borehole fluid evaluation tool to evaluate the characteristics of a fluid through x-ray backscatter imaging is also disclosed, the method including at least producing x-rays in a shaped output; measuring the intensity of backscatter x-rays returning from the fluid to each pixel of one or more array imaging detectors; and converting intensity data from said pixels into characteristics of the wellbore fluids.

### FIELD OF THE INVENTION

The present invention relates generally to the fields of imaging and logging the contents and characteristics of wells, boreholes and hydrocarbon formations, and in a particular though non-limiting embodiment to methods and means of measuring and characterizing fluids disposed within a borehole or pipe containing water, oil or gas, or a mixture thereof.

### BACKGROUND

The oil and gas industry has traditionally classified reservoirs by structures subdivided into geological units or pressure compartments in order to characterize fluids in a formation. Typically, fluid samples from varying depth of the well would either be retrieved from the well or analyzed in situ. Fluid samples which are retrieved require that they are maintained under similar pressures and temperatures of the environment during sampling to ensure that the properties of the sample do not change due to the partial pressures of the contained gases. In situ methods have typically relied upon optical or ultrasonic means for characterizing the fluid in various depths of the well.

Despite the development and advancement of various methods for determining formation fluid properties based on acquiring formation fluid samples from inside the wellbore, there remains a need to provide techniques capable of determining the composition of fluids without altering their properties or physical location by the action of sample collection or interrogation.

There are currently several in-situ methods for fluid recognition available to operators, viz.:
1. Optical fluid characterization;
2. Ultrasonic methods, including time of flight, particulate count and flow rate (Doppler);
3. Electromagnetic methods consisting of irradiation of fluids by gamma rays from radioactive isotopes or x-rays from Bremsstrahlung and detection of transmitted radiation for analysis of the attenuation characteristics of said fluid;
4. Electromagnetic methods consisting of irradiation of fluids by gamma rays from radioactive isotopes or x-rays from Bremsstrahlung and detection of scattered radiation for analysis of the attenuation characteristics of said fluid.

The optical means involves passing key wavelengths of light (from infrared to ultraviolet) through the fluid to determine the attenuation coefficient of said fluid by analyzing the distribution of attenuation characteristics as a function of wavelength. By defining the characteristic distribution signature of the attenuation response to optical wavelengths of a fluid sample, the fluid under interrogation can be compared against a lookup table of known fluid attenuation characteristics and the most probable fluid type determined.

Ultrasonic methods include sending pulses through the fluid between a transducer and a receiver over a predetermined distance, such that the time of flight can be measured and therefore the speed of sound within the fluid determined Additionally, the ultrasonic properties of the fluid may be affected by the particulate content, thus the acoustic signature profile can be used to characterize the particulate content of the fluid. Other means also include using comparative time of flight paths in two directions through a moving fluid such that the Doppler Effect can be measured and the speed of flow of the fluid determined.

Gamma and x-ray transmission methods consist of separating a sample of the fluid from the main borehole and irradiating the sample with gamma or x-rays. A detector placed on the opposite side of the fluid compared to the radiation source then measures the amount and/or spectral energy distribution of the radiation that passes through the sample from said source. The radiation emitted by the source is attenuated in the fluid by and amount dependent upon the electron density profile of the fluid and the energy of the radiation. The resulting radiation transmitted through to the detector thus bears a signature of the composition of the intervening fluid. By comparing the amount and spectral energy distribution of the detected radiation against a database of known materials, the sample fluid can be identified.

Gamma and x-ray scattering methods consist of interrogating a sample of the fluid from the main borehole by irradiating the sample with gamma or x-rays, but without isolating the sample from the main borehole. A detector placed the somewhere adjacent to the radiation source then measures the amount and/or spectral energy distribution of the photons that scatter through the fluid from said source. The radiation emitted by the source is scattered in the fluid by the amount related to the electron density profile of the fluid. Thus, the scattered radiation collected by the detector bears a signature of the composition of the borehole fluid. By comparing the amount and spectral energy distribution of the detected radiation against a database of known materials, the sample fluid can be identified.

The prior art teaches a variety of techniques that use x-rays or other radiant energy to identify or obtain information about the fluid within the borehole of a water, oil or gas well, though none teach any method for interrogating the fluid in front of a tool while said tool is moving through the well as described and claimed later in the application. Such a method provides the benefit that the fluid has not been altered prior to interrogation by mixing, movement of interfaces or otherwise disturbed by passage of the tool.

U.S. Pat. No. 4,980,642A Rodney describes a method for determining the dielectric constant of a fluid within a borehole surrounding a drill pipe. The method uses radar to determine the conductivity of the fluid surrounding the drill pipe within a borehole.

U.S. Pat. No. 4,994,671 A Safinya et al. teaches of a method and means for analyzing the composition of formation fluids through optical spectroscopic methods, employing a comparison between the emitted wavelength of a source light and the detected wavelength after passing through a fluid sample.

U.S. Pat. No. 5,276,656 Angehrn et al. describes a method for using ultrasonic techniques for fluid identification and evaluation in boreholes. The method teaches of a temporal evaluation of the ultrasonic properties of a fluid based on the speed of sound within the fluid, with the aim of determining the volume of said fluid by calculating the rate of change of said fluid properties as a function of volume.

U.S. Pat. No. 4,628,725 Gouilloud et al. describes a method for using ultrasonic techniques for fluid identification and evaluation in a tubular conduit, specifically those surrounding a drill string. The method teaches of a means to determine ultrasonic properties of a fluid based on the speed of sound within the fluid.

U.S. Pat. No. 4,947,683A Minear et al. describes an apparatus which employs a Doppler borehole fluid measuring scheme. A rotating ultrasonic head is described that would be capable of measuring the interfaces between fluid types within a borehole. The concept of measurements based on the Doppler Effect measurements being possible by the flow of gas bubbles within the fluid is also discussed.

U.S. Pat. No. 7,675,029 Teague et al. provides an apparatus that permits the measurement of x-ray backscattered photons from any solid surface inside of a borehole that refers to two-dimensional imaging techniques. It teaches of the possibility for spectroscopy and comparison with a materials database to determine the composition of the materials within the solid surface. However, it fails to teach of a method for determining the fluid type in the borehole itself.

U.S. Pat. No. 8,511,379B Spross et al. describes a system and method for determining properties of a fluid based on the x-ray absorption of a fluid. The concept of transmission absorption with respect to a fluid is taught in addition to a multi-pixel array detector system which is employed to detect tracers within the fluid. However, it fails to teach of a system which combines all of the counts of each pixel such that the overall statistical noise can be reduced, thereby improving the quality of the signal.

U.S. Pat. No. 7,807,962 B2 Waid et al. describes a system and method for determining properties of a fluid based on nuclear magnetic electromagnetic energy absorption of a fluid. The concept of transmission absorption with respect to a fluid is taught in addition to a system for guiding formation fluids from a pad into an assaying tubular section for sample analysis.

U.S. Pat. No. 4,490,609 A Chavalier discloses a method and apparatus for analyzing well fluids through irradiation by x-rays that aims to reduce the effects of the metal casing, cement and/or formation. Dual photon energy bands are used to independently measure the absorption from Thompson scattering and photoelectric effect. However, in the apparatus disclosed by Chavlier, measurements are made in the central section of the apparatus, thus the fluid must be displaced around the tool itself before measurement. However, Chavalier does not teach of a method which does not disturb the fluid prior to or at the time of measurement, as the fluid would already have been displaced around the tool housing itself.

U.S. Pat. No. 2,261,539 Egan et al describes a method and apparatus for analyzing well fluids through irradiation of said fluids by gamma rays from an isotope. In similarity to Chavalier, the detected counts are as a result of the attenuation of the source photons in the annular fluids between the tool and the borehole wall. However, Egan does not teach of a method which does not disturb the fluid prior to or at the time of measurement, as the fluid would already have been displaced around the tool housing itself.

U.S. Pat. No. 7,075,062 B2 Chen et al. describes a system and method for determining properties of a fluid based on x-ray irradiation of a borehole fluid and attenuation measurements. The concept of the link between Compton scattering measurements and the electron density is discussed as well as the possibility of using multiple energy bands. In addition, to a system for guiding formation fluids from a pad into an assaying section of the apparatus for sample analysis, the concept of removal of spurious data resulting from sand influx into the system is also considered.

U.S. Pat. No. 7,507,952 B2 Groves et al. describes a system and method for determining properties of a fluid based on the x-ray absorption of the fluid. The concept of transmission absorption with respect to a fluid is taught in addition to the concept of a fluid comparator cell.

There is, therefore, a long-felt need that remains unmet despite many prior unsuccessful attempts to achieve a forward-looking fluid analysis method that does not seek to remove a sample of fluid from the wellbore into an apparatus or otherwise disturb the fluid. In such a method, the radiation source and imaging device are both located within the tool housing at the lowest point of the apparatus, such that the fluid remains undisturbed and outside of the apparatus during measurement, in a manner that overcomes the various shortcomings of the prior art. In addition, the prior art fails to teach of a mechanism through which an operator can anticipate what fluid changes are about to take place in front of the tool prior to the tool reaching the interface—this gives the operator a pre-warning of the status of the borehole, through the fluid composition of the borehole, in advance of the tool arriving at the sampled location.

### SUMMARY

An x-ray-based borehole fluid evaluation tool for evaluating the characteristics of a fluid located external to said tool in a borehole using x-ray backscatter imaging is provided, the tool including at least an x-ray source; a radiation shield to define the output form of the produced x-rays into the borehole fluid outside of the tool housing; at least one collimated imaging detector to record x-ray backscatter images; sonde-dependent electronics; and a plurality of tool logic electronics and power supply units.

A method of using an x-ray-based borehole fluid evaluation tool to evaluate the characteristics of a fluid through x-ray backscatter imaging is also provided, the method including at least producing x-rays in a shaped output; measuring the intensity of backscatter x-rays returning from the fluid to each pixel of one or more array imaging detectors; and converting intensity data from said pixels into characteristics of the wellbore fluids.

### BRIEF DESCRIPTION OF THE DRAWINGS

FIG. 1 depicts a first embodiment comprising a tool [101] disposed within a fluid [105,109] filled conduit [100] containing an x-ray source [102] which is illuminating a volume of fluid [105,109] separated by a fluid interface [108] with x-rays [104]. The scattered radiation [106] resulting from the x-rays interaction with the fluid [105] located in front of the tool is collected by a detector array [103] or arrays.

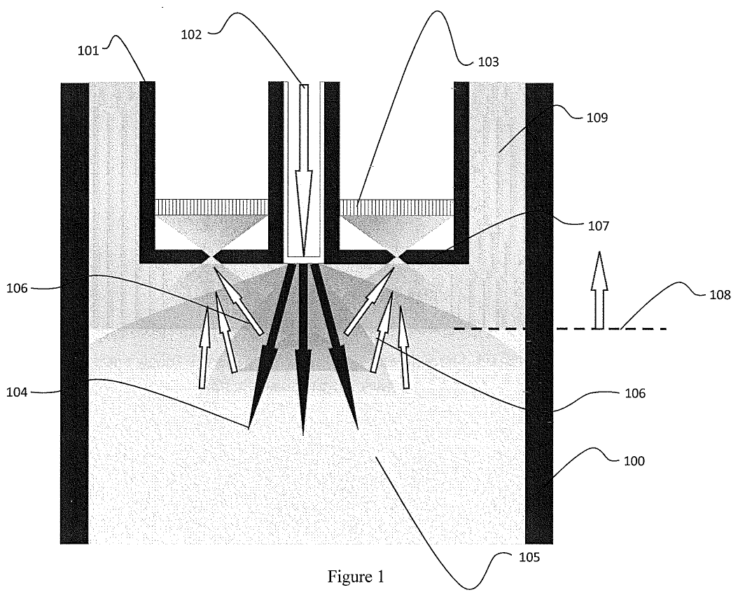

FIG. 2 depicts the same embodiment, however the notion that the tool has moved further into the conduit is illustrated by the movement of the fluid interface [202] into the fluid annulus between the tool and the conduit wall, consequently the scattering response of the fluid [201] will be different to the response of the interface between the two fluids in front of the tool.

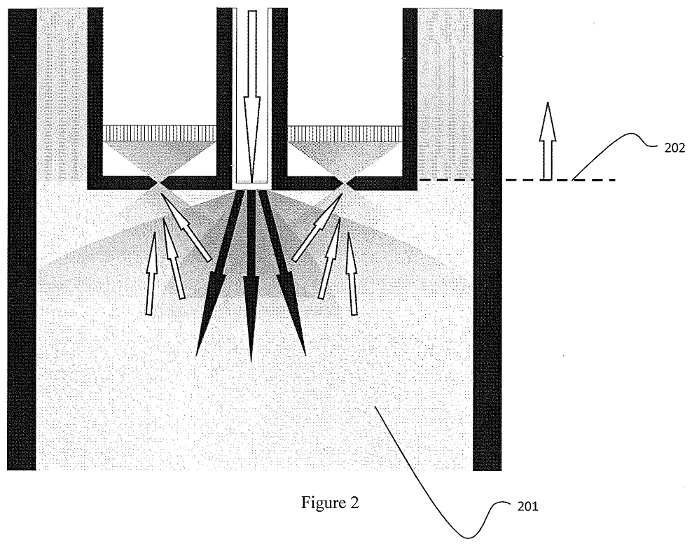

### DETAILED DESCRIPTION OF SEVERAL EXAMPLE EMBODIMENTS

There are no previously known technologies available on the market capable of providing an operator with non-disruptive means for determining the composition of a fluid or location of a fluid interface within a borehole with any significant level of detail with respect to the precise depth of a fluid interface or change.

The invention described and claimed herein therefore comprises a method and means for permitting an operator to determine the precise depth location and characteristic of a fluid in a conduit through a forward-looking fluid analysis method that does not seek to remove a sample of fluid from the wellbore into the apparatus, so that the fluid remains undisturbed and outside of the apparatus during measurement particularly in a region in front of the apparatus as it is lowered in to the conduit.

The objects of the invention are achieved by acquiring radiation backscattered from fluids disposed in front of the tool in the area of the conduit that has not been disturbed by the action of the measurement. The backscattered radiation is to be collected by detector arrays and analyzed in detail using computational comparative characterization techniques.

In the first embodiment, primary x-rays [104] are produced by and x-ray tube [102] located within a pressure resistance tool housing [101]. The primary x-rays illuminate a section of the well fluids [105, 109] in front of the tool [101] and results in the backscattering of radiation from both the Thompson and Compton effects. The scattered radiation [106] enters a collimation device [107] such as a pinhole, optical slot, array collimator or other collimation means, such as an array collimator, and falls upon a detector array [103] or arrays. The scattered radiation [106] is distributed across the surface of the detector array [103] which may consist of a linear array or an area array.

Compared to a detector based upon a single scintillator crystal- and photomultiplier tube, a pixel array effectively consists of many individual detectors, and as the nature of a collimator will always reduce the number of incoming counts compared to no collimation, it can be envisioned that a pixel array will have a number of key benefits. Firstly, by distributing the total collected number of counts over a number of pixels, the statistical noise associated with each pixel can be reduced when the all of the counts associated with all of the detectors is combined as a single reading. An idealized detector would be capable of producing noise statistics identical to Poissonian distribution, however, by increasing the number of individual detectors measuring an acquisition, it is possible to reduce the overall signal to noise ratio within acceptable standards when considering the short acquisition times required to capture a reasonable data rate when considering that the tool is moving through the fluid and through the conduit. Once all of the individual counts associated with each pixel of each detector has been summed, it can be assumed that the statistical noise has been reduced to such an extent that the useable signal to noise ratio is sufficient to determine changes in the overall acquisitioned count rate such that a sufficient (such as <1%) change in fluid response would be detectable.

As the backscatter response of the fluid can be shown to be independent of the density of the fluid to the lowest order, it is possible to create a characteristic response of each of the fluid types that one would expect to encounter in a fluid filled conduit. In that respect, the measured fluid response can be compared against a database of known fluid responses and the fluid type determined as a function of the depth of the tool as it is moved through the conduit.

In a further embodiment, the tool is stationary and the fluid type is determined as a function of depth, using a combination of casing collar locators and run in depth of the wireline without requiring the tool to be moving through the conduit.

In a further embodiment, the detector is a scintillator crystal which is coupled to a photo multiplier tube or photodiode.

In a further embodiment, the primary radiation [104] is produced by a chemical ionizing radiation source, such as a radioisotope.

In a further embodiment, the counts from each pixel of the detector array are not summed to obtain the total counts incident upon the entire detector, but instead individual pixels or groups of pixels are analyzed. This embodiment capitalizes on the highly localized region of space interrogated by each pixel in order to provide information about the spatial variations in fluid properties across the conduit. Furthermore, the scattered radiation recorded by different pixels or groups of pixels represents scattering through different angles as well as different attenuation path lengths. By comparing the signals received by different pixels with respect to these differences in scattering geometry, additional information can be obtained that may improve the fluid identification.

The foregoing specification is provided for illustrative purposes only, and is not intended to describe all possible aspects of the present invention. Moreover, while the invention has been shown and described in detail with respect to several exemplary embodiments, those of skill in the pertinent arts will appreciate that minor changes to the description and various other modifications, omissions and additions may be made without departing from the scope thereof.

1. An x-ray-based borehole fluid evaluation tool for evaluating the characteristics of a fluid located external to said tool in a borehole using x-ray backscatter imaging, wherein said tool comprises:
an x-ray source;
a radiation shield to define the output form of the produced x-rays into the borehole fluid outside of the tool housing;
at least one collimated imaging detector to record x-ray backscatter images;
sonde-dependent electronics; and
a plurality of tool logic electronics and power supply units.

2. The tool of claim 1, wherein said collimated imaging detector comprises a two-dimensional per-pixel collimated imaging detector array, wherein the imaging array is multiple pixels wide and multiple pixels long.

3. The tool of claim 1, wherein said collimated imaging detector comprises a two-dimensional pinhole-collimated imaging detector array, wherein the imaging array is multiple pixels wide and multiple pixels long.

4. The tool of claim 1, wherein said collimated imaging detector collects information regarding backscattered x-ray energy.

5. The tool of claim 1, wherein said radiation shield further comprises tungsten.

6. The tool of claim 1, wherein said tool is configured so as to permit through-wiring.

7. The tool of claim 1, wherein said tool is combinable with other measurement tools comprising one or more of acoustic, ultrasonic, neutron, electromagnetic and other x-ray-based tools.

8. The tool of claim 1, further comprising a means of conveyance to convey the tool through the borehole.

9. The tool of claim 8, further comprising a depth logging device to log the depth of the tool as it is conveyed through the borehole.

10. The tool of claim 9, further comprising a depth correlation system to correlate said x-ray backscatter images with the depth at which the images were acquired.

11. The tool of claim 1, wherein said tool logic electronics further comprise a means to sum groups of pixels from said at least one imaging detector.

12. The tool of claim 1, further comprising an automated computational x-ray backscatter image conversion system to convert said x-ray backscatter images to fluid characteristics.

13. The tool of claim 4, further comprising an automated x-ray backscatter energy conversion system to convert said x-ray backscatter energy information to fluid characteristics.

14. A method of using an x-ray-based borehole fluid evaluation tool to evaluate the characteristics of a fluid through x-ray backscatter imaging, said method comprising:
producing x-rays in a shaped output;
measuring the intensity of backscatter x-rays returning from the fluid to each pixel of one or more array imaging detectors; and
converting intensity data from said pixels into characteristics of the wellbore fluids.

15. The method of claim 14, further comprising measuring the energy of backscatter x-rays returning from the fluid and converting said energy data into characteristics of the wellbore fluids.

16. The method of claim 14, further comprising measuring the energy of backscattered X-rays returning from the fluid and converting said energy data into characteristics of any wellbore materials or debris.

17. The method of claim 14, further comprising measuring the intensity of backscatter x-rays returning from the fluid to one or more subsets of pixels on one or more array imaging detectors.

18. The method of claim 17, further comprising summing the individual intensity measurements of one or more subsets of pixels comprising groups of pixels.

19. The method of claim 14, further comprising combining other measurement methods comprising one or more of acoustic, ultrasonic, neutron, electromagnetic and/or other x-ray-based methods.

20. The method of claim 14, wherein said characteristics of a fluid comprise one or more of:
the composition of said fluid, the density of said fluid, or the water cut of said fluid.

21. The method of claim 14, further comprising
continuously conveying said x-ray-based borehole fluid evaluation tool through a borehole; recording the depth of said tool versus time; periodically measuring one or more of the intensity and energy of backscatter x-rays returning from the fluid; correlating the periodic backscatter x-ray measurements with depth; and converting each of the depth-correlated periodic x-ray backscatter measurements into characteristics of a fluid to create a log of fluid characteristics versus depth.

22. The method of claim 14, further comprising conveying said x-ray-based borehole fluid evaluation tool to one or more pre-determined depths in a borehole; measuring one or more of the intensity and energy of backscatter x-rays returning from the fluid at each pre-determined depth; recording the depth of said tool at each measurement point; correlating the backscatter x-ray measurements with depth; and converting each of the depth-correlated x-ray backscatter measurements into characteristics of a fluid to create a log of fluid characteristics versus depth.

23. The method of claim 14, further comprising using automated computations to convert backscatter X-ray energy information into fluid characteristics.
